(19) United States
(12) Patent Application Publication
(10) Pub. No.: US 2007/0129665 A1
(43) Pub. Date: Jun. 7, 2007

(54) Nasal drug delivery

(75) Inventors: Colin Dickens, Towcester (GB); Bahman Asgharian, Cary, NC (US); Julia S. Kimbell, Chapel Hill, NC (US); Owen Thomas Price, Cary, NC (US); Geoff Brace, Columbus, NC (US)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: BESPAK PLC, WOLVERTON MILL SOUTH MILTON KEYNES (GB)

(21) Appl. No.: 10/553,330

(22) PCT Filed: Apr. 8, 2004
(86) PCT No.: PCT/GB04/01535

§ 371 Date: Feb. 13, 2007

(30) Foreign Application Priority Data
Apr. 17, 2003 (GB) 0308986.9

(51) Int. Cl. A61M 37/00; A61M 31/00

(52) U.S. Cl. 604/26; 604/48

(57) ABSTRACT

A method and device are disclosed for delivering a drug aerosol to the turbinate region (4, 6, 8) of the nasal passage. The drug is delivered to the nostril in a metered volume of gas such that it reaches the turbinates, and flow into the nostril is then prevented for sufficient time to allow the particles to settle. The particles have an aerodynamic diameter below about 12.5 μm such that the majority pass the nasal valve

This invention relates to the delivery of drugs to the turbinate region of the nose, particularly that of the human body. These can include pain management drugs, vaccines, biologics and hormones, amongst others.

Nasal drug delivery devices are available which deliver an active material or drug to the nasal passages, for example in the form of a spray delivered from a nozzle introduced into the nostril. Thus the drug is commonly provided to the nasal passage as a ‘cloud’ of particles carried in air. For the drug to be absorbed into the bloodstream, it is preferable for the drug particles to deposit in the turbinate region of the nasal passage. It is estimated that current devices achieve on average from 1% to 4% deposition in the turbinate region, with the rest of the particles depositing in the nasal vestibule, or passing into the lungs. This invention aims to increase or maximise deposition in the region of the nose that has been identified as a primary target, and to reduce undesirable effects which are associated with deposition in the lungs.

According to the present invention, there is provided a method of delivering an active material to the tissue of the nasal turbinate region, comprising administering to a nostril the active material in the form of particles small enough to pass the nasal valve in a gas flow, together with a volume of gas to deliver the particles to the turbinate region, and substantially preventing further gas flow through the nostril for a predetermined time period to allow the particles to settle on the tissue. Conveniently the gas is air, and flow is prevented by sealing the nostril. Thus the active material is conveniently delivered from a device which forms a seal with the nostril. Alternatively, the subject can be instructed to hold their breath. The particles may contain a carrier material in addition to the active material, or may be mixed with particles of a carrier material.

Studies have shown that when a particulate drug is inhaled or propelled through the nostril, larger particles (for example those having an aerodynamic diameter of 15 μm and above) mainly deposit in the vestibule and nasal valve, with only a small fraction reaching the turbinates. This is because they have a relatively high momentum and therefore do not change direction easily. Thus they tend to impact on the nasal tissue in the frontal structure of the nose. In contrast, smaller particles (aerodynamic diameter of 5 μm and below) mainly deposit in the lungs, passing both the vestibule and nasal valve, and with only a small proportion depositing in the turbinate region. Therefore the theoretical maximum turbinate deposition which it is believed is achievable with current devices in their standard mode of use is only about 20%, with medium sized particles (i.e. around 7.5 μm to 10 μm).

With the present invention, the particles are sized such that a substantial proportion of the particles are small enough to pass the nasal valve. Thus they preferably have an aerodynamic diameter below about 12.5 μm. The particle cloud is preferably generated by and carried in a volume of air which is sufficient to carry it into but not substantially beyond the turbinate region. Thus, the volume preferably exceeds the volume of the nasal vestibule but does not substantially exceed the combined volume of the nasal vestibule and the turbinate region. The air flow is then substantially halted, such that the particles can deposit on the tissue of the turbinates by sedimentation (that is, gravitational settling). If sufficient time is allowed for all the particles to settle, those which would have deposited in the lungs will deposit instead in the turbinates. In this way the deposition in the turbinates of particles containing active material is substantially increased, and it is estimated that the total deposition of smaller particles (i.e. around 5 μm) could be higher than 80%.

The particles containing the active material may be mixed with particles of a carrier material. For example the carrier material particles may be much larger, for facilitating handling of the mixture of the active and carrier material particles, such as pouring into a delivery device. The larger particles of carrier material may deposit in the nasal vestibule upon delivery, with the particles of active material continuing into the turbinate region.

Furthermore, any reference herein to particles of an active material includes particles containing both an active material and a carrier material. Thus, a given range of particle size may include particles wholly comprised of active material and particles comprised of active material and carrier material. The carrier material may be any pharmaceutically acceptable material, such as lactose or calcium carbonate.

When an active material is processed to produce small particles, the result is usually particles having a distribution of different sizes, typically a log normal distribution. Thus, whilst particles of different sizes may be present in the material delivered, preferably at least about 25% by mass of the active material is provided in particles small enough to pass the nasal valve, more preferably at least about 35% is in such particles, even more preferably at least about 50% is in such particles, still more preferably at least 75% is in such particles, and even more preferably at least 90% is in such particles.

The time required for particles to deposit by sedimentation is related to the particle aerodynamic diameter and the distance which the particles must fall to land on a surface, which in this case is the maximum height of the airways in the turbinates. Small particles settle at a slower rate than larger particles, and larger airways require longer for particles to reach the lower airway surface than smaller airways. Thus the time period is preferably at least about 0.5 seconds, more preferably at least about 1 second, still more preferably at least about 2 seconds, and even more preferably at least about 3 or 4 seconds.

Allowing for variations in nasal sizes, it is assumed that the gas flow in the nostril should be prevented for a sufficient time for particles to settle a minimum distance of about 3 mm, and a maximum distance of about 7 mm, in order for a substantial proportion of them to reach the surface of the turbinates in most noses. Thus, assuming the gas is air, the predetermined time period is preferably at least about 0.5 seconds. Preferably the active material is delivered in particles having an aerodynamic diameter of from about 2.5 μm to about 12.5 μm, and the predetermined time period is from about 30 seconds to about 0.5 seconds.

More preferably, the aerodynamic diameter of the particles is from about 4 μm to about 10 μm, and the predetermined time period is from about 14 second to about 1 second. Still more preferably the aerodynamic particle diameter is from about 5 μm to about 9 μm, and the predetermined time period is from about 9 second to about 1 second. Even more preferably, the aerodynamic particle diameter is from about 6 μm to about 8 μm, and the predetermined time period is from about 6.5 second to about 1.5 seconds.

In preferred examples, and assuming a required settling distance of 5 mm, which is considered to be an average turbinate height, the active material is delivered in particles having an aerodynamic diameter of about 5 μm, and the time period is about 6 seconds; or the aerodynamic particle diameter is about 7.5 μm, and the time period is about 3 seconds.

If a settling distance of 3 mm is required, then the time period is preferably from about 15 to about 0.6 seconds, for particles having an aerodynamic diameter from about 2.5 μm to about 12.5 μm. Preferably when the particles are from about 4 μm to about 10 μm, the time period is from about 6 seconds to about 1 second, when the particles are from about 5 μm to about 9 μm the time period is preferably from about 3.9 to about 1.2 seconds, when the particles are from about 6 μm to about 8 μm, the time period is preferably from about 2.7 to about 1.5 seconds, when the particles are about 5 μm, it is preferably about 3.9 seconds, and when the particles are about 7.5 μm, it is preferably about 1.7 seconds.

If the required settling distance is 5 mm, then the time period is preferably from about 25 second to about 1.0 seconds, for particles from about 2.5 μm to about 12.5 μm. For particles from about 4 μm to about 10 μm, the time period required is from about 10 to about 1.6 seconds, for particles from about 5 μm to about 9 μm the time period required is from about 6.4 to about 2.0 seconds, and for particles from about 6 μm to about 8 μm the time period required is from about 4.5 to about 2.5 seconds.

If the required settling distance is 7 mm, then the time period is preferably from about 35 to about 1.5 seconds, for particles from about 2.5 to about 12.5 μm. For particles from about 4 μm to about 10 μm, the time period required is from about 14 to about 2.3 seconds, for particles from about 5 μm to about 9 μm, the time period required is from about 9 to about 3 seconds, for particles from about 6 μm to about 8 μm the time period required is from about 6.3 to about 3.6 seconds, for 5 μm particles it is about 9 seconds, and for 7.5 μm it is about 4 seconds.

Measurements have shown that the combined volume of the nasal vestibule and the turbinate region in the adult population is between about 1 and about 30 mls. Thus, based on delivery to an adult, the volume of fluid is preferably between 0.5 and 25 mls, and in particular may be between 3 and 15 mls. A more preferred volume is between 6 and 10 mls, and more particularly 5.7 ml.

In some cases, it is undesirable for particles to reach the lungs and therefore it is preferable that there are very few particles present of a very small size. In such cases, the mean particles size may be higher than the optimum size for turbinate deposition, so as to minimise the presence of very small particles. For example, if the mean particle size is 20 μm, then typically only around 10-15% of the particles are smaller than 10 μm, and only about 1% are smaller than 5 μm. This invention then has the advantage that most of any small particles that are present will settle in the turbinates whilst the gas flow is halted. Therefore they are less likely to cause a problem, than when conventional methods are used.

The present invention also provides a method of operating a device for delivering an active material to the tissue of the nasal turbinate region, comprising providing in the device an active material in the form of particles small enough to pass the nasal valve in a gas flow, inserting a nozzle of the device into a nostril such that the nozzle forms a substantially gas-tight seal therewith, actuating the device to deliver the active material together with a volume of gas into the turbinate region, and retaining the seal between the nozzle and the nostril for a predetermined time period to allow the particles to settle on the tissue.

The invention also provides a device for delivering to the tissue of the nasal turbinate region, an active material in the form of particles small enough to pass the nasal valve in a gas flow, comprising a nozzle for insertion into a nostril, the nozzle being arranged to form a substantially gas-tight seal with the nostril, a housing for containing the active material, a delivery means arranged to deliver the material from the nozzle in a volume of gas to the turbinate region, and means for indicating when a predetermined time period has elapsed after actuation of the delivery means, so as to allow the particles to settle on the tissue.

Preferably, the administration device may either deliver a predetermined volume of air entraining the particulate active material, or delivery may be achieved by inhalation of the active material with a predetermined volume of air.

Thus, from another aspect, the invention also provides a device for delivering to the tissue of the nasal turbinate region an active material in the form of particles small enough to pass the nasal valve in a gas flow, comprising a nozzle for insertion into a nostril, the nozzle being arranged to form a substantially gas-tight seal with the nostril, a housing for containing the active material, a delivery means arranged to deliver the material to the nozzle in a gas flow, and means for determining when a predetermined volume of gas has passed through the nozzle to deliver the particles to the turbinate region, and substantially preventing further gas flow through the nozzle thereafter, so as to allow the particles to settle on the tissue.

For example, the device may include means for measuring the inhaled volume, and blocking the flow after the predetermined volume has been inhaled. In one embodiment, this could be achieved by providing a critical orifice through which the gas flow passes, and measuring the time period for which gas flow takes place. This will give approximately the same inhaled volume regardless of inhalation pressure.

Because the nozzle seals with the nostril, if the device is then held in place after actuation, this will block gas flow in the nostril. The subject may still breath through the mouth or through the other nostril during this period. Alternatively, the subject may be instructed to hold their breath for a predetermined time period after actuation of the device.

An embodiment of the invention will now be described by way of example with reference to the accompanying drawings, in which:

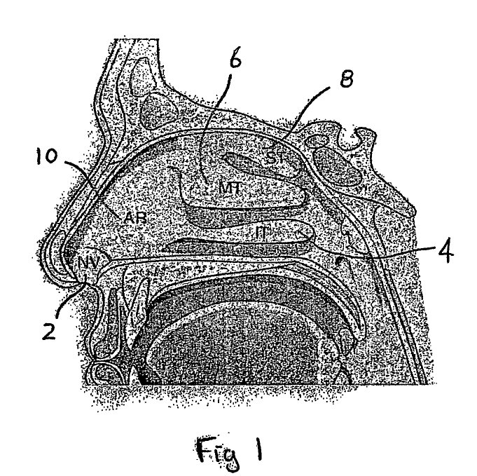

FIG. 1 is a stylised diagram of the nasal anatomy of a human being;

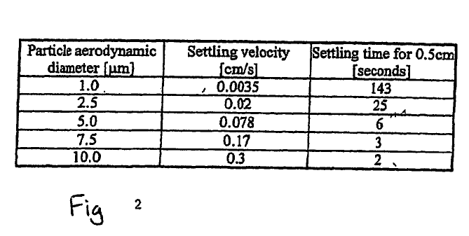

FIG. 2 shows settling times for particles of various aerodynamic diameters;

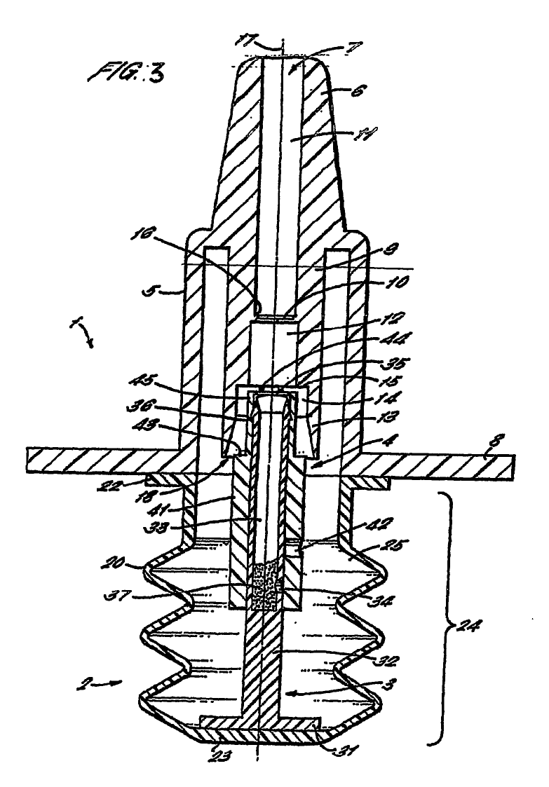

FIG. 3 is a cross sectional view of a device suitable for carrying out the method of the present invention in a “storage” condition; and

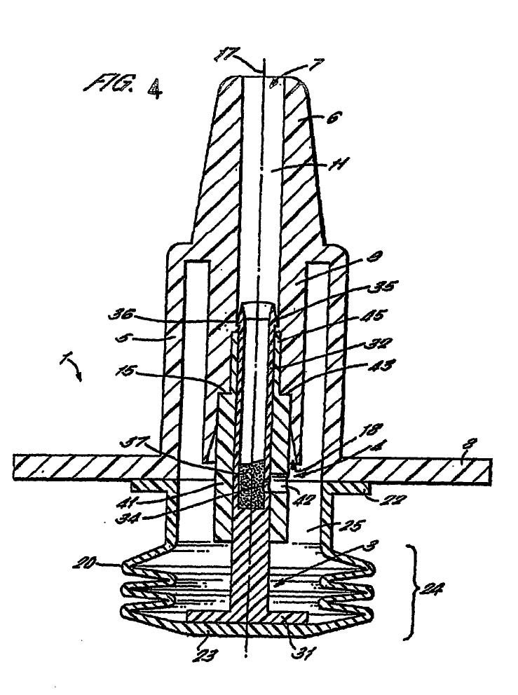

FIG. 4 is a cross sectional view of the device of FIG. 4 in a “dispensing” condition.

Referring to FIG. 1, the nasal passage comprise the following parts. The nasal vestibule 2 is the area directly inside each nostril. The turbinate region includes the inferior turbinate 4, the middle turbinate 6, and the superior turbinate 8. A narrowing of the air passages between the vestibule 2 and the turbinates 4, 6 and 8 is known as the nasal valve 10. The turbinate region 4, 6 and 8 is lined with respiratory epithelium cells, and has a plentiful supply of blood vessels. This tissue is a major target for drug delivery, allowing a quick route into the blood supply.

Drugs for nasal delivery are commonly provided in particulate form. The aerodynamic particle size or “aerodynamic diameter” is a term used in aerosol physics to provide a particle size definition that relates directly to how a particle behaves in a fluid such as air. For non-spherical particles, clearly the term “diameter” is not applicable. For example, the particle may be a flake or a fibre. Moreover particles having the same diameter which are composed of different chemical compounds may have different densities. Thus the aerodynamic diameter is the equivalent diameter of a spherical particle having a density of 1 g per cubic centimetre that has the same inertial properties (i.e. terminal settling velocity) in the fluid as the particle of interest. An inertial sampling device such as a cascade impactor can be used for particle sizing. Such a sampling device can be used to determine the aerodynamic diameter.

FIG. 2 shows the settling velocities for particles by aerodynamic diameter, and settling time required to fall 0.5 cm, which is the estimated average height of an airway passage in the turbinates. For example a cloud of 5 μm particles would require 6 seconds to deposit in the turbinates, with fluid flow through the nostril to which the cloud has been administered being avoided during this period. This can be achieved by a breath hold, or by the delivery device blocking the nostril.

The particle cloud is conveniently propelled from a delivery device by a volume of gas, such as air. In order to target the turbinates, it is necessary for the particle cloud to be held after the equivalent volume of the vestibule has been propelled into the nostril or inhaled, but before the combined equivalent volume of the vestibule and the turbinate has been propelled or inhaled. In practice it is assumed that the particle cloud will be at the front of the propelling volume of gas, for example in the first 2 ml, and will then stop within the turbinate region.

Referring to FIGS. 3 and 4, a suitable administration device is shown. This device is the subject of WO 02/30500, the content of which is incorporated herein by reference. The device comprises a housing 1 having a nozzle 6 with an internal passage 11 having an outlet 7. The nozzle 6 is sized so as to fit into a nostril, and is tapered so as to form a seal therewith. A shaft 32 has a storage chamber 33 therein for containing an active ingredient 37 in particulate form. The storage chamber 33 has an outlet 35 for communicating with the nozzle passage 11. The chamber 33 is housed within a sheath 4 slidably mounted on the shaft, and having a frangible membrane 44 closing an outlet thereof which overlies the outlet 35 of the chamber 33. The sheath 4 has an inlet 42 which is initially offset from an inlet 34 of the chamber 33.

A variable volume actuator 2 is operatively connected to the shaft 33 such that operation of the actuator to reduce the volume and pressurise the gas in an interior 3 of the actuator causes the shaft 33 to move within the sheath 4. The shaft 33 moves from an initial “storage” position to a “dispensing” position in which the inlets of the sheath and the chamber, 42 and 34, are brought into alignment. At the same time, a shoulder 43 of the sheath 4 abuts a step 15 in the internal passage 11 of the housing 1, such that the shaft 32 slides within the sheath 4 and ruptures the frangible membrane 44. Thus a gas flow path is opened from the interior 3 of the actuator through the chamber 33 into the nozzle passage 11 (see FIG. 3). Pressurised gas from the interior 3 of the actuator is then discharged along the gas flow path to entrain the powder material 37 and dispense it through the nozzle outlet 7.

In use, the nozzle 6 is pushed into the nostril until a seal is formed therewith. The device is then actuated by pushing the end 23 of the actuator. The powdered drug is thus dispensed into the nostril followed by a predetermined volume of air corresponding to the internal capacity of the device, such that it reaches the target region of the nasal passage. Devices having different internal capacities may provided depending upon the size of the subject and/or the size of the nasal passage of the subject. Alternatively, the device may have a variable internal capacity.

The nozzle 6 is then held in place for a predetermined period of time to allow the particles to settle onto the tissue, for absorption into the bloodstream. The device may be provided with an indicator of time elapsed from actuation. For example, there may be a visual or audible signal that a predetermined time period has elapsed. This time period may be varied depending again on the size of the subject, and/or the size of the subjects nasal passage, and hence the time required for the particles to settle.

Alternatively, the active material may be inhaled from a delivery device. In this case, there may be an orifice provided between the chamber containing the particulate material and the nozzle. The nozzle is inserted into a nostril, and when the subject inhales through the nozzle, air is drawn through the orifice entraining the material and passing through the nozzle into the nostril. Such a device would include means for measuring the inhaled volume. For example, the orifice may be sized to be a ‘critical orifice’ for air, such that measuring the time during which air flow takes place provides an indication of the inhaled volume, which is relatively independent of inhalation pressure. The device may then shut off the flow after a time which corresponds to the required volume, for example by deploying a physical barrier across the flow passage.

Whilst the invention has been described with reference to gas and in particular air, transporting the active material, it is possible that other fluids could be used.

This invention is suitable for administering any suitable active material. Suitable active ingredients are not limited by therapeutic category, and can be, for example, analgesics, anti-emetics, anti-inflammatory agents, anthelmintics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, beta.-Blockers, calcium channel blockers, cardiac inotropic agents, corticosteroids, diuretics, anti-parkinsonian agents, gastro-intestinal agents, histamine receptor antagonists, keratolytics, lipid regulating agents, anti-anginal agents, cox-2 inhibitors, leucotriene inhibitors, macrolides, muscle relaxants, nutritional agents, opioid analgesics, patassium channel activators, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and mixtures thereof.

Likewise, the active ingredient can be a cytokine, a peptidomimetic, a peptide, a protein, a toxoid, a serum, an antibody, a vaccine, a nucleoside, a nucleotide, a portion of genetic material, a nucleic acid, or a mixture thereof.

Specific, non-limiting examples of suitable active ingredients are: acarbose; acetyl cysteine; acetylcholine chloride; acutretin; acyclovir; alatrofloxacin; albendazole; albuterol; alendronate; alglucerase; amantadine hydrochloride; ambenomium; amifostine; amiloride hydrochloride; aminocaproic acid; aminogluthemide; amiodarone; amlodipine; amphetamine; amphotericin B; antihemophilic factor (human); antihemophilic factor (porcine); antihemophilic factor (recombinant); aprotinin; asparaginase; atenolol; atorvastatin; atovaquone; atracurium besylate; atropine; azithromycin; azithromycin; aztreonam; bacitracin; baclofen; BCG vaccine; becalermin; beclomethsone; belladona; benezepril; benzonatate; bepridil hydrochloride; betamethasone; bicalutanide; bleomycin sulfate; budesonide; bupropion; busulphan; butenafine; calcifediol; calciprotiene; calcitonin human; calcitonin salmon; calcitriol; camptothecan; candesartan; capecitabine; capreomycin sulfate; capsaicin; carbamezepine; carboplatin; carotenes; cefamandole nafate; cefazolin sodium; cefepime hydrochloride; cefixime; cefonicid sodium; cefoperazone; cefotetan disodium; cefotoxime; cefoxitin sodium; ceftizoxime; ceftriaxone; cefuroxime axetil; celecoxib; cephalexin; cephapirin sodium; cerivistatin; cetrizine; chlorpheniramine; cholecalciferol; cholera vaccine; chrionic gonadotropin; cidofovir; cilostazol; cimetidine; cinnarizine; ciprofloxacin; cisapride; cisplatin; cladribine; clarithromycin; clemastine; clidinium bromide; clindamycin and clindamycin derivatives; clomiphene; clomipramine; clondronate; clopidrogel; codeine; coenzyme Q10; colistimethate sodium; colistin sulfate; cortocotropin; cosyntropin; cromalyn sodium; cyclobenzaprine; cyclosporine; cytarabine; daltaperin sodium; danaproid; danazol; dantrolene; deforoxamine; denileukin; diftitox; desmopressin; dexchlopheniramine; diatrizoate megluamine and diatrizoate sodium; diclofenac; dicoumarol; dicyclomine; didanosine; digoxin; dihydro epiandrosterone; dihydroergotamine; dihydrotachysterol; diltiazemi; dirithromycin; domase alpha; donepezil; dopamine hydrochloride; doxacurium chloride; doxorubicin; editronate disodium; efavirenz; elanaprilat; enkephalin; enoxacin; enoxaprin sodium; ephedrine; epinephrine; epoetin alpha; eposartan; ergocalciferol; ergotamine; erythromycin; esmol hydrochloride; essential fatty acid sources; etodolac; etoposide; factor IX; famiciclovir; famotidine; felodipine; fenofibrate; fentanyl; fexofenadine; finasteride; flucanazole; fludarabine; fluoxetine; flurbiprofen; fluvastatin; foscarnet sodium; fosphenytion; frovatriptan; furazolidone; gabapentin; ganciclovir; gemfibrozil; gentamycin; glibenclamide; glipizide; glucagon; glyburide; glycopyrolate; glymepride; GnRH; gonadorelin; gonadotropin releasing hormone and synthetic analogs thereof; granulocyte colony stimulating factor; granulocyte-macrophage stimulating factor; grepafloxacin; griseofulvin; growth hormone-bovine; growth hormones-recombinant human; halofantrine; hemophilus B conjugate vaccine; heparin sodium; hepatitis A virus vaccine inactivated; hepatitis B virus vaccine inactivated; ibuprofen; indinavir sulfate; influenza virus vaccine; insulin aspart; insulin detemir; insulin glargine; insulin lispro; insulin NPH; insulin procine; insulin-human; interferon alpha; interferon beta; interleukin-2; interleukin-3; ipratropium bromide isofosfamide; irbesartan; irinotecan; isosorbide dinitrate isotreinoin; itraconazole; ivermectin; japanese encephalitis virus vaccine; ketoconazole; ketorolac; lamivudine; lamotrigine; lanosprazole; leflunomide; leucovorin calcium; leuprolide acetate; levofloxacin; lincomycin and lincomycin derivatives; lisinopril; lobucavir; lomefloxacin; loperamide; loracarbef; loratadine; lovastatin; L-thryroxine; lutein; lycopene; mannitol; measles virus vaccine; medroxyprogesterone; mefepristone; mefloquine; megesterol acetate; meningococcal vaccine; menotropins; mephenzolate bromide; mesalmine; metformin hydrochloride; methadone; methanamine; methotrexate; methoxsalen; methscopolamine; metronidazole; metroprolol; mezocillin sodium; miconazole; midazolam; miglitol; minoxidil; mitoxantrone; mivacurium chloride; montelukast; mumps viral vaccine; nabumetone; nalbuphine; naratiptan; nedocromil sodium; nelfinavir; neostigmine bromide; neostigmine methyl sulfate; neutontin; nicardipine; nicorandil; nifedipine; nilsolidipine; nilutanide; nisoldipine; nitrofurantoin; nizatidine; norfloxacin; octreotide acetate; ofloxacin; olpadronate; omeprazole; ondansetron; oprevelkin; osteradiol; oxaprozin; oxytocin; paclitaxel; pamidronate disodium; pancuronium bromide; paricalcitol; paroxetine; pefloxacin; pentagastrin; pentamindine isethionate; pentazocine; pentostatin; pentoxifylline; periciclovir; phentolamine mesylate; phenylalanine; physostigmine salicylate; pioglitazone; piperacillin sodium; pizofetin; plague vaccine; platelet derived growth factor-human; pneumococcal vaccine polyvalent; poliovirus vaccine inactivated; poliovirus vaccine live (OPV); polymixin B sulfate; pralidoxine chloride; pramlintide; pravastatin; prednisolone; pregabalin; probucol; progesterone; propenthaline bromide; propofenone; pseudo-ephedrine; pyridostigmine; pyridostigmine bromide; rabeprazole; rabies vaccine; raloxifene; refocoxib; repaglinide; residronate; ribavarin; rifabutine; rifapentine; rimantadine hydrochloride; rimexolone; ritanovir; rizatriptan; rosigiltazone; rotavirus vaccine; salmetrol xinafoate; saquinavir; sertraline; sibutramine; sildenafil citrate; simvastatin; sincalide; sirolimus; small pox vaccine; solatol; somatostatin; sparfloxacin; spectinomycin; spironolactone; stavudine; streptokinase; streptozocin; sumatriptan; suxamethonium chloride; tacrine; tacrine hydrochloride; tacrolimus; tamoxifen; tamsulosin; targretin; tazarotene; telmisartan; teniposide; terbinafine; terbutaline sulfate; terzosin; tetrahydrocannabinol; thiopeta; tiagabine; ticarcillin; ticlidopine; tiludronate; timolol; tirofibran; tissue type plasminogen activator; tizanidine; TNFR:Fc; TNK-tPA; topiramate; topotecan; toremifene; tramadol; trandolapril; tretinoin; trimetrexate gluconate; troglitazone; trospectinomycin; trovafloxacin; tubocurarine chloride; tumor necrosis factor; typhoid vaccine live; ubidecarenone; urea; urokinase; valaciclovir; valsartan; vancomycin; varicella virus vaccine live; vasopressin and vasopressin derivatives; vecoronium bromide; venlafaxine; vertoporfin; vigabatrin; vinblastin; vincristine; vinorelbine; vitamin A; vitamin B12; vitamin D; vitamin E; vitamin K; warfarin sodium; yellow fever vaccine; zafirlukast; zalcitabine; zanamavir; zidovudine; zileuton; zolandronate; zolmitriptan; zolpidem; zopiclone; and pharmaceutically acceptable salts, isomers and derivatives thereof; and mixtures thereof.

In particular, it is envisaged that the active material may comprise pain management drug such as Sumatriptan, Zolmitriptan, Dihydroergotamine (migraine), Butorphanol (break through pain), hormones such as Desmopressin acetate (diabetes insipidus/polyuria), Calcitonin—salmon (Hypercalcaemia, Paget's disease), oxytocin (control labour, bleeding and milk secretion), naferelin & buserelin (emdometriosis, CCP), nicotine and vitamin B12 (pernicious aneamia).

Other drugs specifically thought to be suitable for intra-nasal delivery are lobeline, deslorelin, vardenafil, insulin, glucagon, oxycodone, pumactant, apomorphine, lidocaine +dextromethorphane, ketamine, morphine, fentanyl, pramorelin, ondansteron, interferon alpha, interferon beta, scopolamine, vomeropherin, alprazolam, triazolam, midazolam, parathyroid hormone, growth hormone, GHRH, somatostatin, melatonin and several experimental NCEs, and vaccines such as these for E coli, Streptococcus A, Influenza, Parainfluenza, RSV, Shigella, Heliobacter Pylori, Versinia pestis, AIDS, Rabies, Periodontitis, and Anti-arthritic vaccines.

It is also contemplated that the vaccines and biologics may be administered in accordance with the invention.

1. A method of delivering an active material to the tissue of the nasal turbinate region, comprising administering to a nostril the active material in the form of particles small enough to pass the nasal valve in a gas flow, together with a volume of gas to deliver the particles into the turbinate region, and substantially preventing further gas flow through the nostril for a predetermined time period to allow the particles to settle on the tissue.

2. A method as claimed in claim 1, in which the particles have an aerodynamic diameter below about 12.5 μm.

3. A method as claimed in claim 1, in which the volume of gas exceeds the volume of the nasal vestibule but does not substantially exceed the combined volume of the nasal vestibule and the turbinate region.

4. A method as claimed in claim 1, wherein the active material is delivered from a device which forms a substantially gas tight seal with the nostril.

5. A method as claimed in claim 1, in which predetermined time period is at least about 0.5 seconds.

6. A method as claimed in claim 5, in which the predetermined time period is at least about 1 second.

7. A method as claim in claim 6, in which the predetermined time period is at least about 2 seconds.

8. A method as claimed in claim 7, in which the predetermined time period is at least about 3 seconds.

9. A method as claimed in claim 8, in which the predetermined time period is at least about 4 seconds.

10. A method as claimed in claim 5, in which the particles have an aerodynamic diameter of from about 2.5 μm to about 12.5 μm, and the predetermined time period is from about 30 second to about 0.5 seconds.

11. A method as claimed in claim 10, in which the particles have an aerodynamic diameter from about 4 μm to about 10 μm, and the predetermined time period is from about 14 seconds to about 1 second.

12. A method as claimed in claim 11, in which the particles have an aerodynamic diameter from about 5 μm to about 9 μm, and the predetermined time period is from about 9 seconds to about 1 second.

13. A method as claimed in claim 12, in which the particles have an aerodynamic diameter from about 6 μm to about 8 μm, and the predetermined time period is from about 6.5 second to about 1.5 seconds.

14. A method as claimed in claim 12, wherein the particles have an aerodynamic diameter of about 5 μm, and the time period is about 6 seconds.

15. A method as claimed in claim 13, wherein the particles have an aerodynamic diameter of about 7.5 μm, and the time period is about 3 seconds.

16. A method as claimed in claim 1, in which less than 15% of the active material is provided in particles having an aerodynamic diameter below about 10 μm.

17. A method as claimed in claim 1, in which at least about 25% by mass of the active material is provided in particles of the defined size.

18. A method as claimed in claim 17, in which at least about 35% by mass of the active material is provided in particles of the defined size.

19. A method as claimed in claim 18, in which at least about 50% by mass of the active material is provided in particles of the defined size.

20. A method as claimed in claim 19, in which at least about 75% by mass of the active material is provided in particles of the defined size.

21. A method as claimed in claim 20, in which at least about 90% by mass of the active material is provided in particles of the defined size.

22. A method as claimed in claim 1 in which the volume of gas is between about 0.5 ml and about 30 mls.

23. A method as claimed in claim 22, in which the volume of gas is between about 2 mls and about 25 mls.

24. A method as claimed in claim 23, in which the volume of gas is between about 3 mls and about 15 mls.

25. A method as claimed in claim 24, in which the volume of gas is between about 6 mls and about 10 mlis.

26. A method as claimed in claim 25, in which the volume of gas is about 5.7 ml.

27. A method of operating a device for delivering an active material to the tissue of the nasal turbinate region, comprising providing in the device an active material in the form of particles small enough to pass the nasal vale in a gas flow, inserting a nozzle of the device into a nostril such that the nozzle forms a substantially gas-tight seal therewith, actuating the device to deliver the active material together with a volume of gas into the turbinate region, and retaining the seal between the nozzle and the nostril for a predetermined time period to allow the particles to settle.

28. A device for delivering to the tissue of the nasal turbinate region an active material in the form of particles small enough to pass the nasal valve in a gas flow, comprising a nozzle for insertion into a nostril, the nozzle being arranged to form a substantially gas-tight seal with the nostril, a housing for containing the active material, a delivery means arranged to deliver the material from the nozzle in a volume of gas to the turbinate region, and means for indicating when a predetermined time period has elapsed after actuation of the delivery means, so as to allow the particles to settle on the tissue.

29. A device for delivering to the tissue of the nasal turbinate region an active material in the form of particles small enough to pass the nasal valve in a gas flow, comprising a nozzle for insertion into a nostril, the nozzle being arranged to form a substantially gas-tight seal with the nostril, a housing for containing the active material, a delivery means arranged to deliver the material to the nozzle in a gas flow, and means for determining when a predetermined volume of gas has passed through the nozzle to deliver the particles to the turbinate region, and substantially preventing further gas flow through the nozzle thereafter, so as to allow the particles to settle on the tissue.

30. A device as claimed in claim 27, in which the volume of gas exceeds the volume of the nasal vestibule but does not substantially exceed the combined volume of the nasal vestibule and the turbinate region.
